(19) United States
(12) Patent Application Publication
(10) Pub. No.: US 2015/0290347 A1
(43) Pub. Date: Oct. 15, 2015

(54) STERILIZATION FOR NEEDLELESS CONNECTORS

(71) Applicants: Samuel Braden, Salt Lake City, UT (US); Eric Peterson, Salt Lake City, UT (US)

(72) Inventors: Samuel Braden, Salt Lake City, UT (US); Eric Peterson, Salt Lake City, UT (US)

(21) Appl. No.: 14/682,371

(22) Filed: Apr. 9, 2015

Related U.S. Application Data
(60) Provisional application No. 61/977,097, filed on Apr. 9, 2014.

(51) Int. Cl. A61L 2/10; A61M 39/16

(52) U.S. Cl. CPC A61L 2/10; A61M 39/16; A61M 2039/1077

(57) ABSTRACT

An adapter for sterilizing a needleless connector that has interior and exterior surfaces. The adapter includes a proximal portion that is coupleable in electromagnetic radiation receiving communication with an electromagnetic radiation source and a cavity portion that is coupleable with the needleless connector and that is shaped to receive at least a portion of the needleless connector. The proximal portion and the cavity portion are made from a translucent material so that electromagnetic radiation from the electromagnetic radiation source can, upon operation, propagate through and be refracted by the translucent material to sterilize both the interior and the exterior surfaces of the needleless connector.

### CROSS-REFERENCE TO RELATED APPLICATION

This application claims the benefit of U.S. Provisional Patent Application No. 61/977,097, filed Apr. 9, 2014, which is incorporated herein by reference.

### FIELD

The subject matter of the present disclosure relates generally to sterilization devices. More specifically, this application relates to sterilization devices for medical devices.

### BACKGROUND

Sterilization and decontamination of medical equipment is an important aspect of providing safe medical care. Conventional practices for sterilizing and decontaminating medical equipment generally involve wiping medical equipment with alcohol fluids, volatile solvents, antiseptic solutions, or disinfectant compositions, among others.

While these conventional practices may be effective at treating and killing certain pathogens, other pathogens and infectious agents may remain active on the medical equipment due to a pathogen's resistance to a particular decontamination solvent or due to non-uniform, uneven, or inconsistent application of the particular decontamination solvent by a practitioner.

### SUMMARY

From the foregoing discussion, it should be apparent that a need exists for a sterilization apparatus and method that overcome the limitations of conventional sterilization practices. Beneficially, such an apparatus and method would improve the ease, efficiency, and effectiveness of sterilization practices for re-usable medical equipment.

The subject matter of the present application has been developed in response to the present state of the art, and in particular, in response to the problems and needs in the art that have not yet been fully solved by currently available sterilization practices. Accordingly, the present disclosure has been developed to provide a sterilization apparatus and method that overcome many or all of the above-discussed shortcomings in the art.

According to one embodiment, disclosed herein is an adapter for sterilizing a needleless connector that has interior and exterior surfaces. The adapter includes a proximal portion that is coupleable in electromagnetic radiation receiving communication with an electromagnetic radiation source and a cavity portion that is coupleable with the needleless connector and that is shaped to receive at least a portion of the needleless connector. The proximal portion and the cavity portion are made from a translucent material so that electromagnetic radiation from the electromagnetic radiation source can, upon operation, propagate through and be refracted by the translucent material to sterilize both interior and exterior surfaces of the needleless connector.

In one implementation, the translucent material is quartz, other silica-derived materials, or fluoropolymers. According to another implementation, the cavity portion may include a male luer-lock fitting. Also, the cavity portion may be shaped to have an annular ridge, an annular trench, and a protrusion, with the annular trench between the annular ridge and the protrusion. In such an implementation, as the needleless connector is coupled to the cavity portion of the adapter, the protrusion actuates a valve mechanism of the needleless connector to further expose the interior surfaces of the needleless connector to the electromagnetic radiation. Further, as the valve mechanism is actuated, the interior surfaces of the needleless connector are isolated from atmosphere external to the adapter. In another implementation, a distal surface of the annular ridge has a first surface roughness, and respective surfaces of the annular trench and the protrusion have a second surface roughness, the first surface roughness being lower than the second surface roughness.

Also disclosed herein is one embodiment of an apparatus for sterilizing a needleless connector. The apparatus includes a housing that has an opening, an electromagnetic radiation source positioned in the housing, and an adapter coupled to the housing and spanning the opening. The electromagnetic radiation source is operable to emit electromagnetic radiation and the adapter includes a cavity portion that is coupleable with the needleless connector and that is shaped to receive at least a portion of the needleless connector. The adapter is made from a translucent material and the electromagnetic radiation propagates through and is refracted by the translucent material to sterilize both interior and exterior surfaces of the needleless connector.

In one implementation, the cavity portion includes an annular trench that defines an annular ridge that is radially external to the annular trench and a protrusion that is radially internal to the annular trench. In such an implementation, a disposable tip may be detachably coupleable over the opening of the housing, with the disposable tip being shaped to complement and contour distal surfaces of the cavity portion and to engage a portion of an external surface of the housing adjacent the opening.

In one implementation, the disposable tip may have a first region and a second region that have different electromagnetic radiation properties. For example, the first region may substantially block the electromagnetic radiation and the second region may substantially transmit the electromagnetic radiation. In such an example, the first region may be positioned adjacent a distal surface of the annular ridge and adjacent the portion of the external surface of the housing and the second region may be positioned adjacent surfaces of the annular trench and surfaces of the protrusion.

In one implementation, a distal surface of the annular ridge is polished and surfaces of the annular trench and the protrusion are unpolished and are therefore comparatively rougher. In another implementation, the adapter is made from a first the translucent material and the disposable tip is made from a second translucent material that is different than the first translucent material.

In one implementation, upon coupling the needleless connector to the cavity portion of the adapter, the protrusion actuates a valve mechanism of the needleless connector to further expose the interior surfaces of the needleless connector to the electromagnetic radiation. In such an implementation, the interior surfaces of the needleless connector may remain isolated from external atmospheric contamination due to the mechanical coupling between the adapter and the needleless connector.

According to another implementation, the translucent material is quartz, other silica-derived materials, or fluoropolymers. The cavity portion of the adapter may include a male luer-lock fitting. Also, the electromagnetic radiation source may be an ultraviolet light source and the electromagnetic radiation may be ultraviolet light that is non-visible to a human-eye. The apparatus may further include a visible light source that is coupled to the housing that emits visible light when ultraviolet light is emitted from the ultraviolet light source in order to indicate operation and thus emission of ultraviolet light (since ultraviolet light is not within the visible spectrum).

In one implementation, the apparatus may further include a connection sensor that verifies a proper connection between the needleless connector and the cavity portion of the adapter. The apparatus may also include a controller that prevents emission of electromagnetic radiation from the electromagnetic radiation source unless the connection sensor verifies the proper connection.

Also disclosed herein is one embodiment of a method for sterilizing a needleless connector. The method includes providing an electromagnetic radiation source positioned within a housing, with the housing having an opening. The method further includes providing an adapter that is coupled to the housing and that spans the opening. The adapter may have a cavity portion that is shaped to receive at least a portion of the needleless connector and the adapter may be made from a translucent material. The method further includes coupling the needleless connector to the cavity portion of the adapter and subsequently emitting electromagnetic radiation from the electromagnetic radiation source for a period of time to sterilize both interior and exterior surfaces of the needleless connector. During emission, electromagnetic radiation propagates through and is refracted by the translucent material of the adapter. After emitting the electromagnetic radiation for the period of time, the method includes decoupling the needleless connector from the cavity portion of the adapter.

According to one implementation, the method further includes verifying, by a connection sensor and a controller, a proper connection between the needleless connector and the cavity portion of the adapter before emitting the electromagnetic radiation. In another implementation, the period of time is set by the controller.

Reference throughout this specification to features, advantages, or similar language does not imply that all of the features and advantages that may be realized with the present disclosure should be or are in any single embodiment of the invention. Rather, language referring to the features and advantages is understood to mean that a specific feature, advantage, or characteristic described in connection with an embodiment is included in at least one embodiment of the subject matter disclosed herein. Thus, discussion of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize that the subject matter of the present application may be practiced without one or more of the specific features or advantages of a particular embodiment. In other instances, additional features and advantages may be recognized in certain embodiments that may not be present in all embodiments of the disclosure. Further, in some instances, well-known structures, materials, or operations are not shown or described in detail to avoid obscuring aspects of the subject matter of the present disclosure. These features and advantages of the present disclosure will become more fully apparent from the following description and appended claims, or may be learned by the practice of the disclosure as set forth hereinafter.

### BRIEF DESCRIPTION OF THE DRAWINGS

In order that the advantages of the disclosure will be readily understood, a more particular description of the disclosure briefly described above will be rendered by reference to specific embodiments that are illustrated in the appended drawings. Understanding that these drawings depict only typical embodiments of the disclosure and are not therefore to be considered to be limiting of its scope, the subject matter of the present application will be described and explained with additional specificity and detail through the use of the accompanying drawings, in which:

FIG. 1 is a perspective view of an apparatus for sterilizing needleless connectors, according to one embodiment;

FIG. 2 is a side view of the apparatus, according to one embodiment;

FIG. 3 is a cross-section side view of the apparatus, according to one embodiment;

FIG. 4 is an exploded side view of the apparatus, according to one embodiment;

FIG. 5 is an exploded cross-section side view of the apparatus, according to one embodiment;

FIG. 6 is a cross-section side view of the apparatus for sterilizing needleless connectors, according to one embodiment;

FIG. 7 is an exploded cross-section side view of the apparatus, according to one embodiment;

FIG. 8 is a frontal perspective view of an adapter for sterilizing needleless connectors, according to one embodiment;

FIG. 9 is a rearward perspective view of the adapter of FIG. 8, according to one embodiment;

FIG. 10 is a frontal perspective view of a disposable tip coupleable to the adapter, according to one embodiment;

FIG. 11 is a rearward perspective view of the disposable tip of FIG. 10, according to one embodiment;

FIG. 12 is a perspective view of a needleless connector, according to one embodiment;

FIG. 13 is a side view of the needleless connector of FIG. 12, according to one embodiment;

FIG. 14 is a cross-section side view showing an apparatus for sterilizing needleless connectors and a needleless connector decoupled from the apparatus, according to one embodiment;

FIG. 15 is a cross-section side view showing the needleless connector of FIG. 14 coupled directly to an adapter of the apparatus of FIG. 14, with a valve mechanism of the needleless connector in an open position, according to one embodiment;

FIG. 16 is a cross-section side view showing an apparatus for sterilizing needleless connectors and a needleless connector coupled to a disposable tip of the, according to one embodiment; and

FIG. 17 is a schematic flowchart diagram of a method of using an apparatus to sterilize a needleless connector, according to one embodiment.

### DETAILED DESCRIPTION

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

In the following description, numerous specific details are provided. One skilled in the relevant art will recognize, however, that the subject matter of the present application may be practiced without one or more of the specific details, or with other methods, components, materials, and so forth. In other instances, well-known structures, materials, or operations are not shown or described in detail to avoid obscuring aspects of the disclosure.

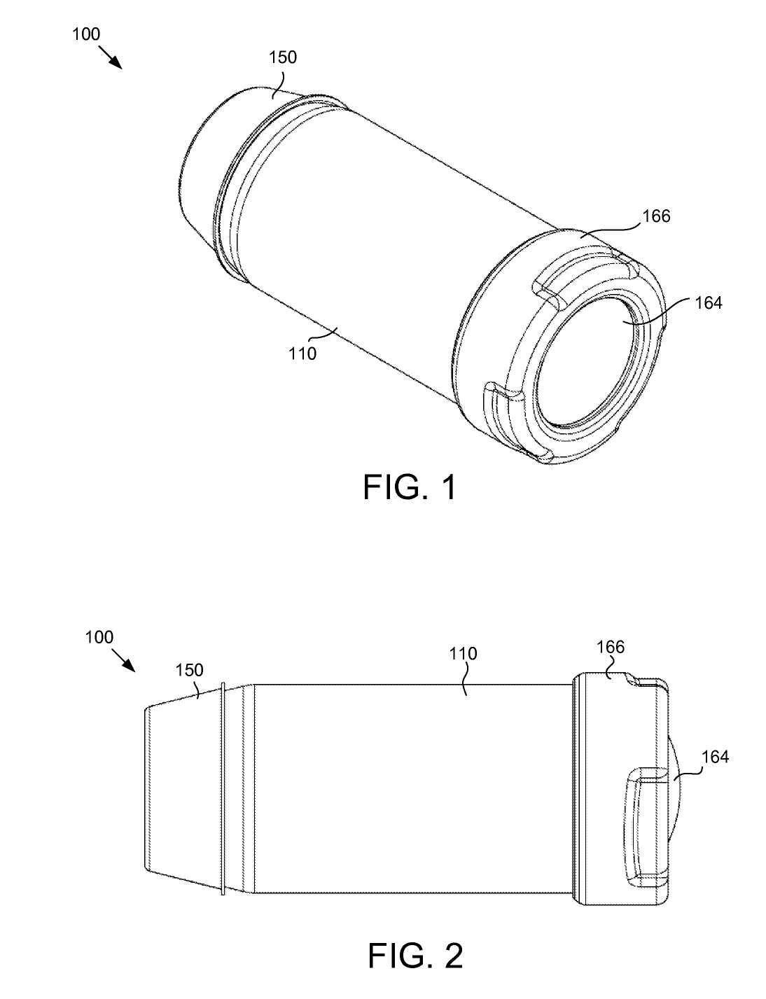

Illustrated in FIGS. 1-17 are several representative embodiments of an apparatus for sterilizing needleless connectors, which embodiments also include one or more methods of sterilizing needleless connectors. As described herein, the apparatus provides several significant advantages and benefits over other sterilization tools. However, the recited advantages are not meant to be limiting in any way, as one skilled in the art will appreciate that other advantages may also be realized upon practicing the present disclosure.

Generally, a practitioner may physically couple a needleless connector to the apparatus 100 and may utilize the apparatus 100 to sterilize and decontaminate at least the portion of the needleless connector that is physically coupled to the apparatus 100. The apparatus 100 utilizes electromagnetic radiation to sterilize and decontaminate the needleless connector. In other words, since certain wavelengths of electromagnetic radiation are bactericidal and are lethal to pathogens, the sterilizing device 100 emits electromagnetic radiation that propagates through an adapter and contacts the needleless connector.

In one embodiment, the apparatus 100 includes a housing 110, an electromagnetic radiation source 120, and an adapter 130. The apparatus 100 may further include a power source 162, various circuitry components 163, a switch 164, and a housing cap 166. The housing 110 houses the electromagnetic radiation source 120 and has an opening 112. The adapter 130 is coupled to the housing 110 and spans the opening 112 and the needleless connector may be coupled directly to the adapter 130. The adapter 130 is made from a translucent material and electromagnetic radiation from the electromagnetic source 120 propagates through and is refracted by the translucent material to sterilize both interior and exterior surfaces of the needleless connector. In one embodiment, the emission of electromagnetic radiation from the electromagnetic radiation source 120 can be controlled via the switch 164. In a further embodiment, the apparatus may also include a disposable tip 150 that is coupleable over the opening 112 of the housing 110. The disposable tip 150 may provide various benefits, such as protecting the adapter 130 from physical wear and tear, maintaining sterilization of the adapter 130 between uses, preventing cross-contamination of the adapter 130 (i.e., swapping disposable tips 150 before sterilizing a needleless connector of a different patient), modifying the transmission of electromagnetic radiation through the adapter 130, and changing the manner of coupling the needleless connector to the apparatus 100, etc. All of these components, as well as various additional components, are described in detail throughout the disclosure and with reference to various embodiments shown in the figures.

In one embodiment, the apparatus 100 may be configured to resemble a pen. For example, apparatus 100 may include clip that facilitates secure engagement of the apparatus 100 within the pocket of a practitioner or that facilitates secure engagement of the apparatus 100 to a clipboard or patient file.

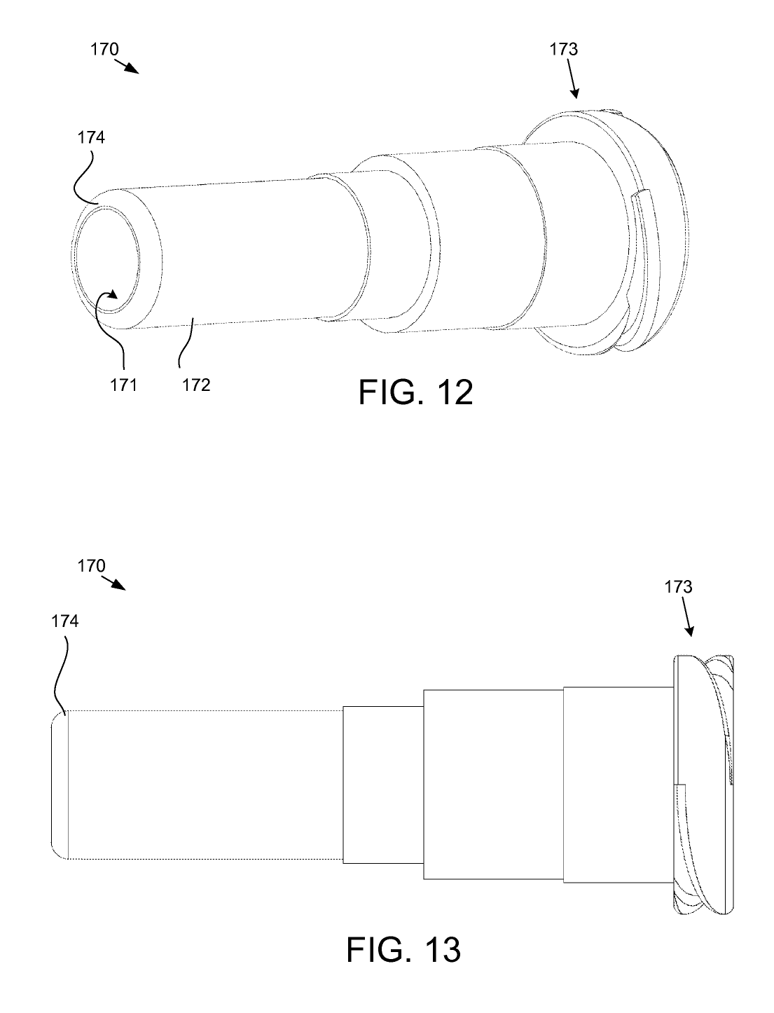

As defined herein, the term “needleless connector” (see FIGS. 12 and 13 for additional details) refers to a reusable segment of medical equipment that is able to connect with medical devices, medical supplies, or other medical equipment without using a needle/septum configuration. For example, in one embodiment the term “needleless connector” refers to the connecting portion of a fluid line, such as an intravenous catheter (“IV” line), a peripherally inserted central catheter (“PICC” line), etc., that is connectable with a fluid supply or a fluid receptacle. Since most fluid lines are designed to be used for the administration and delivery of fluid from various sources (e.g., repeat doses from IV bags, etc.) or the withdrawal of body fluid to various receptacles, the connecting portion is used to repeatedly connect and disconnect from various types of medical equipment. Thus, the term “needleless connector” does not necessarily preclude the inclusion of a needle-type connector downstream (from a fluid injecting perspective) of the needleless connector. Additional details relating to the needleless connector and the associated means for repeatedly connecting to various types of medical equipment are included below with reference to FIGS. 12 and 13.

The housing 110 may be cylindrical and have a circular cross-section, as depicted, or the housing 110 may have rectangular, elliptical, triangular, or other polygonal cross-sections. The housing 110 may be made from metallic materials, polymer materials, composite materials, etc. In one embodiment, the housing 110 is opaque and thus does not allow the transmission of electromagnetic radiation through the walls of the housing 110. In one embodiment, the interior surface 116 of the housing 110 may be reflective or may have a reflective coating. However, in one embodiment the interior surface 116 of the housing 110 may be specifically shaped so as to not resemble a parabolic reflector in order to prevent a focused beam of electromagnetic radiation. In other words, the interior surfaces 116 of the housing 110 may contribute to a distributed emission of electromagnetic radiation in substantially different directions, thus promoting complete sterilization and decontamination of both interior and exterior surfaces of the needleless connector.

In one embodiment, the housing 110 may have a frusto-conical tip, with the surface at the tapered end of the frusto-conical tip defining the opening 112 of the housing 110. However, in another embodiment the housing 110 may have a uniform cross-sectional along its entire length or may not have a narrowing/tapering tip that defines the opening 112.

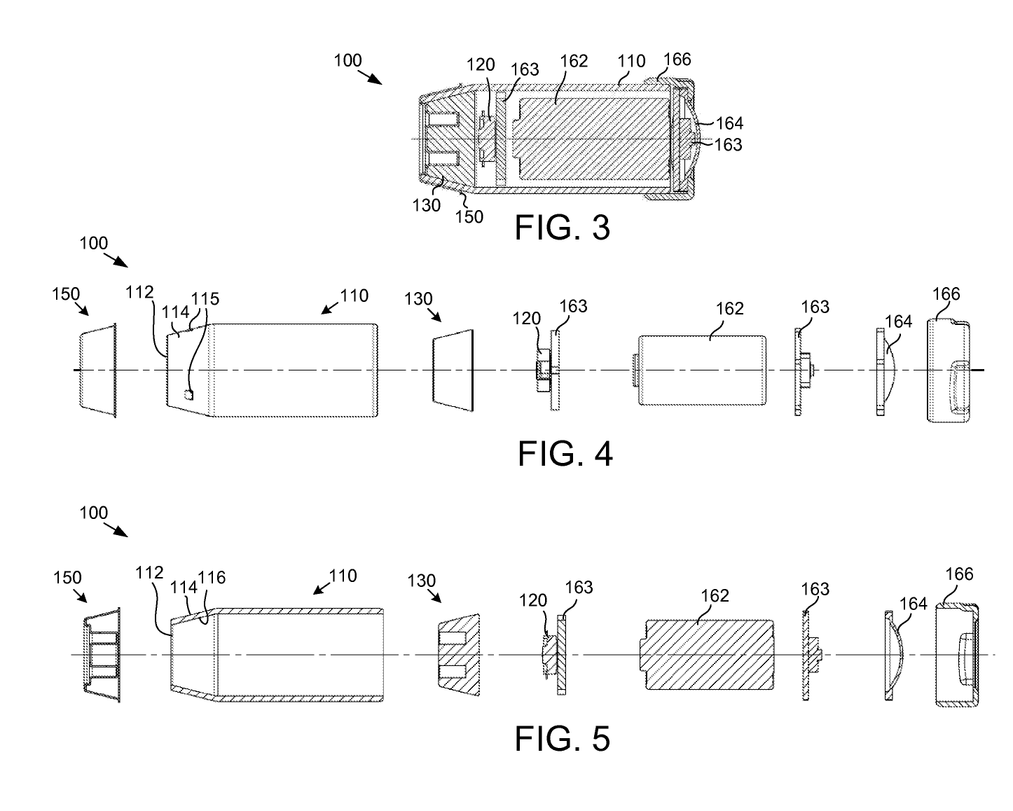

Referring to FIGS. 3-5, the power source 162 may be a disposable/replaceable power source, such as a battery. In another embodiment, the power source 162 may be a permanently installed battery that may be rechargeable. In yet another embodiment, the apparatus 100 may include a power source interface that is electrically connectable to a remote power source (e.g., an electrical outlet).

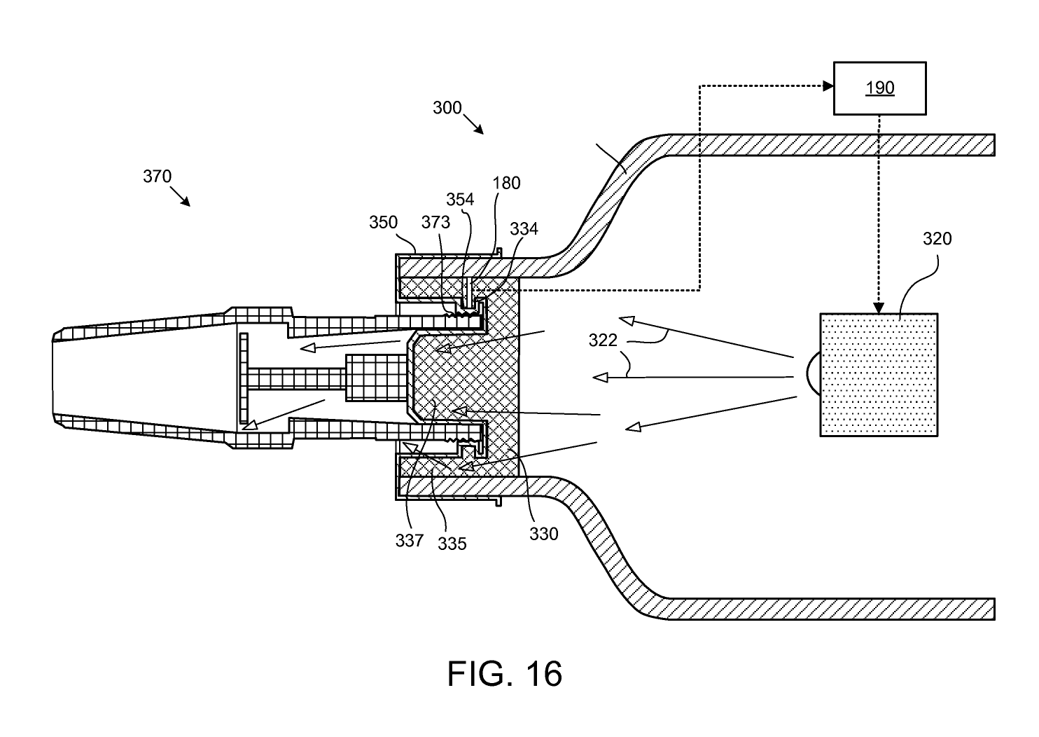

The switch 164 may be electrically connected between the power source 162 (e.g., a battery) and the electromagnetic radiation source 120 via the various circuitry components 163 in order to turn the electromagnetic radiation source 120 on or off. The switch 164 may be disposed on an end of the apparatus 100 that is opposite the opening 112. In another embodiment, the switch 164 may be disposed on a circumferential surface of the housing 110. In one embodiment the switch 164 may be a simple on/off type toggle switch. In another embodiment, the switch 164 may be a hold-and-release type switch that requires a practitioner to hold a button in a depressed position during the duration of the emission of electromagnetic radiation from the electromagnetic radiation source 120. In a further embodiment, the switch 164 may allow the practitioner to select from a plurality of predetermined electromagnetic radiation intensities, thus allowing the practitioner to customize the sterilization procedure. In yet another embodiment, emission of electromagnetic radiation may be automated using a controller (see below with reference to FIG. 16).

The apparatus 100 may include other features, such as a detachable and/or retractable hood that blocks and shields practitioners and patients from inadvertent/stray electromagnetic radiation exposure and retains such electromagnetic radiation within the device for further sterilization. A retractable hood may also facilitate mechanical coupling between the needleless connector and the adapter 130 and may also serve to turn off the electromagnetic radiation source should it be retracted prematurely during treatment. A retractable hood may also utilize a twist and lock mechanism to ensure it stays in place during treatment. The apparatus 100 may also optionally include a sterilizing fluid injector that, in conjunction with the sterilizing electromagnetic radiation from the electromagnetic radiation source 120, facilitates decontamination of the needleless connector. A portion of the housing 110, a portion of the adapter 130, or a portion of the retractable hood may be made from a material that is reactive to certain types of electromagnetic radiation such that the material illuminates or changes color to indicate to the practitioner that the device is functioning as intended.

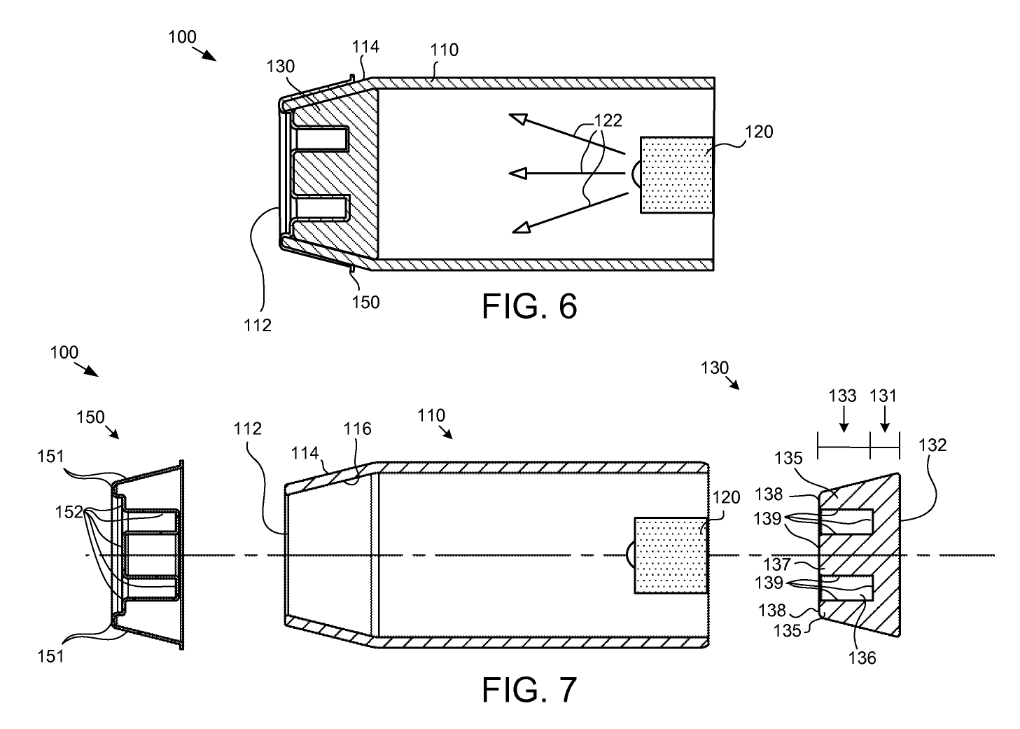

As mentioned above, the apparatus 100 may include a disposable tip 150. The disposable tip 150 may be coupled to the housing 110 using engagement features 115 that are disposed on the external surface 114 of the housing 110. In other words, the disposable tip 150 may include features that correspond with the engagement features 115, thus securing the disposable tip 115 to the housing 110. In one embodiment, the engagement features 115 may include a series of protrusions on the external surface 114 of the housing 110 that engage corresponding indentations on the internal surface of the disposable tip 150. In another embodiment, the disposable tip 150 may be coupled to the housing 110 using an interference fit or a friction fit. Further details relating to function and structure of the disposal tip 150 are included below with reference to FIGS. 6-11.

The adapter 130, although described in greater detail below with reference to FIGS. 6-9, is made from a translucent material through which electromagnetic radiation can propagate and may include features that allow the needleless connector to be mechanically coupled to the adapter 130. In one embodiment, the adapter 130 is permanently coupled to the housing 110. For example, the adapter 130 may be mounted and secured within the housing 110 using adhesives, bonding compositions, physical retaining features, etc. In another embodiment, the adapter 130 may be detachably coupled to the housing 110, thus allowing the adapter 130 to be replaced or removed for repair. In one embodiment, the adapter 130 is housed within and substantially confined by the housing 110. In another embodiment, however, the adapter 130 may at least partially extend through the opening 112 or may extend beyond the confines of the housing 110. In other words, the adapter 130 is positioned and oriented relative to the opening 112 of the housing 110 such that any electromagnetic radiation that passes through the opening 112 will also pass through the adapter 130. Once again, additional details relating to the adapter 130 are included below.

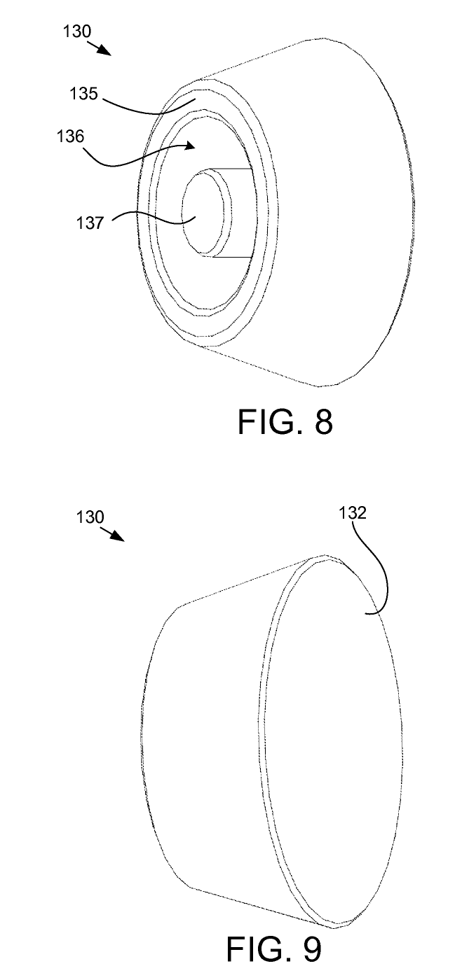

FIGS. 6-7 show various views of a representative embodiment of an apparatus 100 used for sterilizing at least a portion of the needleless connector and FIGS. 8-9 show various views of a representative embodiment of the adapter 130 used for sterilizing at least a portion of the needleless connector. More specifically, FIG. 6 is a longitudinal cross-section view and FIG. 7 is an exploded longitudinal cross-section view of the apparatus 100 while FIG. 8 is a front perspective view and FIG. 9 is a back perspective view of the adapter 130.

As shown in FIG. 6, electromagnetic radiation 122 emitted from the electromagnetic radiation source 120 passes through the adapter 130 before contacting a needleless connector coupled to the adapter 130. As mentioned above, certain wavelengths of electromagnetic radiation 122 are effective at killing pathogens. For example, in one embodiment the electromagnetic radiation source 120 is an ultraviolet light source and correspondingly the electromagnetic radiation 122 is ultraviolet light (e.g., light with wavelength between about 10 nanometers and about 400 nanometers). In another embodiment, the electromagnetic radiation source 120 may emit other wavelengths of electromagnetic radiation to sterilize the needleless connector. For example, light that has a wavelength of greater than 400 nm, which may be visible to certain people, may also have germicidal/bactericidal properties. The electromagnetic radiation source 120 may be positioned proximate the adapter 130 (as shown in FIG. 3) or the electromagnetic radiation source 120 may be positioned a distance away from the adapter 130.

In one embodiment, the adapter 130 is made from a translucent material that allows electromagnetic radiation (e.g., ultraviolet light) to be transmitted through the adapter 130. For example, the adapter 130 may be made from silica-derived materials, such as quartz (e.g., fused-quartz), or other similar material that transmits and refracts ultraviolet light, thus allowing ultraviolet light to propagate through the adapter. In another embodiment, silica derivatives or polymers may be used as the material of the adapter 130. For example, polymers such as polytetrafluoroethylene (“PTFE”), fluorinated ethylene propylene (“FEP”), and other fluoropolymers may be used as the material of the adapter 130. Such polymers may be translucent only for certain wavelengths of electromagnetic radiation (e.g., ultraviolet light).

In one embodiment, the adapter 130 is not completely transparent and at the same time is not completely opaque. In other words, in one embodiment the adapter 130 may prevent the straight-through passage of electromagnetic radiation 122 (i.e., induces refraction) and/or may partially diminish the perceived radiant flux of the electromagnetic radiation 122 (by diffusing and refracting the radiation) without completely blocking transmission of electromagnetic radiation 122. In one implementation, the apparatus 100 may also include a visible light source that admits visible light when ultraviolet light is emanating from the ultraviolet light source, thus providing visible feedback and visible indication of the status of the non-visible, ultraviolet light. The visible light source may be disposed external to the housing so that a practitioner might easily see the visible light indication.

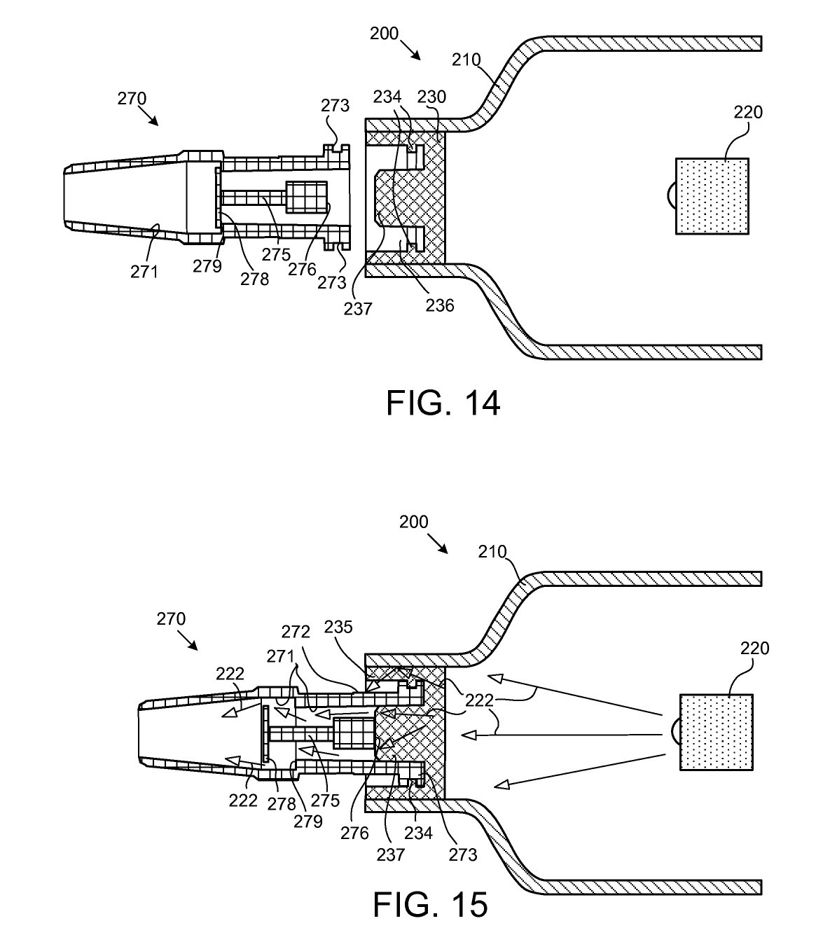

As mentioned above, the electromagnetic radiation 122 propagating through the adapter 130 is configured to be refracted and dispersed so that upon exiting the adapter 130 the radiation contacts both interior and exterior surfaces of the needleless connector. Additional details regarding propagation and refraction of the electromagnetic radiation 122 through the adapter 130 and the sterilizing effect of electromagnetic radiation 122 on the needleless connector are included below with reference to FIGS. 14-16.

In one embodiment, the adapter 130 is a unitary, monolithic structure. For example, the adapter 130 may be solid and may not have any pass-through apertures or pass-through channels. In other words, in one embodiment the propagation and transmission of electromagnetic radiation 122 through the adapter 130 is not the result of discrete conduits or physical channels in the adapter 130 through which light can emanate. Thus, the adapter 130 may form a solid partition through which all of the electromagnetic radiation 122 being emitted from the opening 112 of the housing 110 must pass through.

In one embodiment, the adapter 130 has a proximal portion 131 and a cavity portion 133. Generally, the proximal portion 131 is the solid region of the adapter 130 that is disposed comparatively closer to the electromagnetic radiation source 120 while the cavity portion 133 is the region of the adapter 130 that is comparatively further away from the electromagnetic radiation source 120. The proximal portion 131 has a proximal surface 132 through which electromagnetic radiation 122 emanating from the electromagnetic radiation source 120 passes upon entering the adapter 130 and the cavity portion 133 is the portion of the adapter 130 two which the needleless connector is coupled. In one embodiment, the proximal surface 132 of the proximal portion 131 may not be planar and may instead be contoured (e.g., convex or concave) to contribute to a desired radiation propagation pattern. As described above, the optional disposable tip 150 may be excluded and the needleless connector may be coupled directly to the cavity portion 133 of the adapter 130. However, in another embodiment, the disposable tip 150 may be coupled directly to the cavity portion 133 of the adapter with the needleless connector being coupled directly to the disposable tip 150.

The shape and configuration of the cavity portion 133 of the adapter 130 may contribute to the manner/mechanism of coupling the needleless connector to the adapter 130 and the shape and configuration of the cavity portion 133 of the adapter 130 may also contribute to the propagation and refraction of electromagnetic radiation 122 exiting the adapter 130 and contacting the needleless connector. Similarly, the shape and configuration of the disposable tip 150 may also contribute to the mechanism of coupling between the needleless connector in the adapter 130 and the refraction electromagnetic radiation 122 upon exiting the adapter 130.

In one embodiment, the cavity portion 133 of the adapter 130, for example, has an annular trench 136 that defines an annular ridge 135 and a protrusion 137. In other words, the annular ridge 135 is the section of the cavity portion 133 of the adapter 130 that is radially external to the annular trench 136 and the protrusion 137 is the section of the cavity portion 133 of the adapter 130 that is radially internal to the annular trench 136 (see FIGS. 8 and 9). In one embodiment, the surfaces 139 of the annular trench 136 may include features that facilitate coupling a needleless connector to the adapter 130. For example, in one embodiment the cavity portion 133 of the adapter 130 may include a male luer-lock fitting (see FIGS. 14-16) that is engageable with a corresponding female luer-lock fitting of a needleless connector. In another embodiment, the annular trench 136 of the cavity portion 133 of the adapter 130 may include threads or other fastening features that facilitate coupling the needleless connector to the adapter 130.

In one embodiment, the protrusion 137 may be shaped and configured to engage a valve mechanism (e.g., an internal valve plunger) of a needleless connector. For example, upon coupling a needleless connector to the cavity portion 133 of the adapter 130, the protrusion 137 may be partially inserted within the needleless connector to depress and thereby open a valve mechanism within the needleless connector, thus further exposing the interior surfaces of the needleless connector to electromagnetic radiation. Further details regarding sterilization, needleless connectors, and internal valve mechanisms of the needleless connectors, are included below with reference to FIGS. 14-16.

In one embodiment, various surfaces of the adapter 130 may be machined or polished to have different surface roughness. Properties of transmission and refraction of electromagnetic radiation through a translucent material may be at least partially dependent upon the characteristics and orientations of the surfaces of the translucent material. For example, comparatively rougher surfaces may increase the extent and degree of refraction that occurs as the electromagnetic radiation passes through such surfaces while a comparatively smoother surface may decrease the extent or degree of refraction and may in fact contribute to the reflection of electromagnetic radiation. Accordingly, the refraction and diffusion of electromagnetic radiation 122 through the adapter 130 may be modified and manipulated by controlling the surface roughness of the adapter 130. For example, in one embodiment a distal surface 138 of the annular ridge 135 has a first surface roughness, and respective surfaces of the annular trench and the protrusion have a second surface roughness, the first surface roughness being lower than the second surface roughness. The surface roughness may be changed via mechanical polishing (e.g., the distal surface 138 of the annular ridge 135 may be polished while surfaces 139 of the annular trench 136 and surfaces 139 of the protrusion 137 may be unpolished). In such an embodiment, transmission of electromagnetic radiation 122 through the distal surface 138 of the annular ridge 135 may be prevented or at least diminished, thus decreasing the amount of electromagnetic radiation 122 that exits the adapter 130 without contacting the needleless connector. Conversely, the comparatively rougher/unpolished surfaces 139 promote further refraction, thus improving the diffusion of electromagnetic radiation to the needleless connector.

Although not shown in the figures or explicitly described herein, the cavity portion 133 of the adapter 130 may have other shapes and configurations. In one embodiment, the cavity portion 133 may not have an annular trench that defines a central protrusion but instead may be a recess formed in the adapter 130 that includes features to which the needleless connector may be coupled. In another embodiment, the cavity portion 133 may include an annular trench but the trench may not be circular and may instead be rectangular, triangular, etc. according to the characteristics and shape of the specific needleless connectors. Also, the comparative, relative elevation between the protrusion 137 and the annular ridge 135 may change depending on the specifications of a certain type of needleless connector.

As mentioned above, the disposable tip 150, in certain embodiments, may be detachably coupled to the housing 110 about the opening 112 during a sterilization procedure. The disposable tip 150 may be shaped to complement and contour the surfaces 138, 139 of the cavity portion 133 of the adapter 130 and to engage at least a portion of the external surface 114 of the housing 110 adjacent the opening 112. In one embodiment, the disposable tip 150 may also contribute to the transmission and refraction of electromagnetic radiation 122. For example, the disposable tip 150 may have a first region 151 and a second region 152 that each has different light transmission properties. The first region 151 may prevent or at least diminish transmission of electromagnetic radiation (i.e., reflect) and may be positioned adjacent the distal surface 138 of the annular ridge 135 and adjacent the portion of the external surface 114 of the housing 110. The second region 152 may transmit and refract electromagnetic radiation and may be positioned adjacent the surfaces 139 of the annular trench 136 and the protrusion 137. The first and second regions 151, 152 may be different materials that are bonded together to form the disposable tip 150 or the first and second regions 151 may be different sections of a unitary disposable tip 150 that have been manipulated (physically, chemically, etc.) to have different light transmission properties.

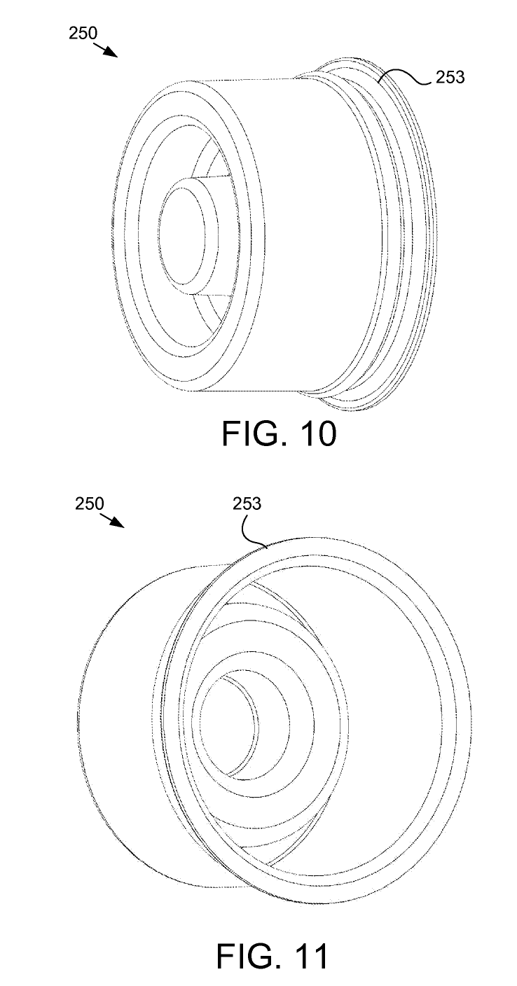

FIGS. 10 and 11 are front and back perspective views, respectively, of another embodiment of the disposable tip 250 that is coupleable to the adapter. The embodiment of the disposable tip 250 depicted in FIGS. 10 and 11 is cylindrical instead of conical. Thus, the disposable tip 250 depicted in FIGS. 10 and 11 is configured to be detachably coupled about the opening in a housing that is cylindrical (see FIGS. 14-16 for depictions of housings that are cylindrical proximate the opening). The disposable tip 250 may have a lip 253 on its proximal end. The lip 253 may allow a practitioner to easily grasp and replace the disposable tip 250. As previously mentioned, the disposable tip 250 may be detachably coupled with the apparatus for various purposes. In one embodiment, the disposable tip 250 is a cap that protects the adapter when the apparatus is not in use. In another embodiment, the disposable tip 250 may be made from translucent material and may be coupled to the adapter/housing when the apparatus is being used to sterilize the needleless connector. For example, the disposable tip 250 may be replaced before using the apparatus on a needleless connector of a different patient, thereby prevent any possibility of cross-contamination between patients.

As mentioned above, the disposable tip 250 may be shaped to complement, match, and contour the cavity portion of the adapter. In one embodiment, the shape of the disposable tip 250 may tightly correspond with the shape of the cavity portion of the adapter, thus eliminating any spatial gaps between the adapter and the disposable tip. In another embodiment, however, spatial gaps between the disposable tip 250 and the adapter may be beneficial in order for the disposable tip 252 to adequately protect the adapter for physical impacts when not in use. Spatial gaps may also be included to provide a contrast in medium, including air, which may reduce the emission of electromagnetic radiation through those surfaces or otherwise affect propagation and refraction of electromagnetic radiation.

FIG. 12 is a perspective view of one embodiment of the needleless connector 170 and FIG. 13 is a side view of the needleless connector 170. As described above, the needleless connector 170 is a reusable segment of medical equipment that is able to connect with medical devices, medical supplies, or other medical equipment without using a needle/septum configuration. The needleless connector 170 may be detachably coupled to tubing, catheters, or other components at its distal end 174 or the needleless connector 170 may be integrally joined and unitary with tubing, catheters, or other components at its distal end 174. The needleless connector 170 may be made from plastic, composite, or other similar material. The needleless connector 170 has an interior surfaces 171 and an exterior surfaces 172. Generally, the interior surfaces 171 of the needleless connector 170 is the interior channel/conduit through which fluid going to or coming from a patient flows. As described below, the needleless connector 170 may include an internal valve mechanism.

The exterior surfaces 172 of the needleless connector 170 may include coupling elements 173. As mentioned above, the coupling elements 173 correspond with the cavity portion 133 of the adapter 130 (or the disposable tip 150). In one embodiment, for example, the coupling elements 173 on the needleless connector 170 may be luer-lock features that engage and securely couple to corresponding luer-lock features on the adapter 130. In another embodiment, the coupling elements 173 may be threads that engage corresponding threads on the adapter 130. In a further embodiment, the needleless connector 170 may be physically coupled to the adapter 130 using an interference/friction fit. Regardless of the exact manner/mechanism of the coupling between the needleless connector 170 and the apparatus 100, the apparatus 100 is configured to physically couple to the needleless connector 170, thereby eliminating the need for the practitioner to hold the needleless connector in close proximity to the apparatus 100. In other words, the apparatus 100 enables the practitioner performing the sterilizing procedure to have at least one-hand free during the sterilization procedure. Further, as described below in greater detail, the physical coupling between the needleless connector 170 and the apparatus 100 contributes to the efficiency and effectiveness of the sterilization procedure by securing the position of the needleless connector 170 with respect to the apparatus 100 in a fixed, known orientation, thus allowing for uniform, repeated, and consistent sterilization of different needleless connectors including dialysis catheters, medical lines, tubes, drains, etc.

FIG. 14 is a longitudinal cross-section view showing the needleless connector 270 uncoupled from the apparatus 200 and FIG. 15 shows the needleless connector 270 coupled directly to the adapter 230 of the apparatus 200. The needleless connector 270 shown in FIGS. 14 and 15 includes an internal valve plunger 275, with the internal valve plunger 275 being in a closed position in FIG. 14 and the internal valve plunger 275 being in an open position in FIG. 15. As described above, the protrusion 237 may engage an engagement surface 276 of the internal valve plunger 275 and may depress the internal valve plunger 275 so that the valve disk 278 is moved distance away from the valve seat 279, thereby further exposing the interior surfaces 271 of the needleless connector 270 to electromagnetic radiation 222. While most needleless connectors are translucent and will allow at least partial transmission of electromagnetic radiation 222 through the walls of the needleless connector 270 to the interior surfaces 271, opening the valve mechanism 275 of the needleless connector 270 allows the electromagnetic radiation 222 to travel comparatively further into the interior surfaces 271 of the needleless connector 270, thus increasing the sterilization and decontamination effect of the electromagnetic radiation 222. Also shown is the electromagnetic radiation 222 impacting and sterilizing exterior surfaces 272 of the needleless connector 270.

As mentioned above, the adapter 230 may include coupling features 234 that correspond with coupling elements 273 on the needleless connector 270. In one embodiment, the coupling features 234 may be disposed on and protrude from lateral walls of the annular trench 236. For example, threads or a male luer-lock fitting may be disposed on the lateral walls of the annular trench 236. In one embodiment, the physical coupling between the adapter 230 and the needleless connector 270 may be such that the atmosphere internal to the needleless connector 270 remains isolated from the ambient atmosphere (atmosphere external to the needleless connector). In one embodiment, even when the needleless connector 270 includes an internal valve mechanism 275 and the valve mechanism to 75 is opened by the protrusion 237, the internal atmosphere of the needleless connector 270 remains isolated from the external ambient atmosphere, thus decreasing the likelihood of contamination from pathogens in the external ambient atmosphere.

FIG. 16 is a longitudinal cross-section view showing the needleless connector 370 coupled to the disposable tip 350 of the apparatus 300, according to one embodiment. As mentioned above, one of the potential uses/purposes of the disposable tip 350 may be to change the means/mechanism by which the needleless connector 370 is coupled to the apparatus 300. In one embodiment, as shown in FIG. 16, the disposable tip 350 may include a dual-sided coupling configuration 354. The dual-sided coupling configuration 354, for example, may include a first-type of coupling element disposed on a first side of the disposable tip 350 (e.g., a female luer-lock fitting) and a second-type of coupling element disposed on a second side of the disposable tip 350 (e.g., threads). The first-type of coupling element (e.g., female luer-lock fitting) may be coupleable with a corresponding coupling feature 334 of the adapter 330 (e.g., a male luer-lock fitting) and the second-type of coupling element (e.g., threads) may be coupleable with corresponding coupling features 373 of the needleless connector 370. In other words, the disposable tip 350 may allow flexibility regarding the type and/or size of needleless connectors 370 that may be coupled to and sterilized by the sterilizing device 300.

The apparatus 300 may also include a controller 190 that automates or controls emission of electromagnetic radiation 322 from the electromagnetic radiation source 320. For example, the duration, intensity, and/or frequency of the emission of electromagnetic radiation 322 may be controlled by the controller 190. The controller may be customizable by a practitioner or different versions of the apparatus 300 may be pre-configured with pre-determined operating parameters depending on anticipated use. As mentioned above, the controller 190 may include a timer module that controls the duration of the emission of electromagnetic radiation 322 based on known sterilization requirements for a specific needleless connector or a specific type of needleless connectors. For example, in one embodiment the controller 190 may actuate emission of electromagnetic radiation for a predetermined period of time between about 0.5 seconds and about 5 minutes. In another embodiment, the controller 190 may actuate emission of electromagnetic radiation for a predetermined period of time between about 15 seconds and about 1 minute. In yet another embodiment, the controller 190 may actuate emission of electromagnetic radiation for a period of time between about 20 seconds and about 40 seconds. The controller 190 may also include a module to regulate the power supply to the electromagnetic radiation source 320 in order to increase or decrease power as needed. In a further embodiment, the controller 190 may also include a module that regulates and controls electromagnetic radiation emission from the electromagnetic radiation source 320 based on one or more of detected radiation intensity, battery life, etc. For example, such a module may disable the apparatus if battery or light source levels drop below predetermined levels.

In one embodiment, the apparatus 300 may include a connection sensor 180 that detects if a proper connection has been made between the needleless connector 370 and the apparatus 300 (directly to the cavity portion of the adapter 330 or directly to the deposable tip 350). The connection sensor 180 may communicate with the controller 190 and the controller may prevent emission of electromagnetic radiation 322 from the electromagnetic radiation source until a proper connection has been verified. The apparatus 300 may further include other components, such as a digital display (e.g., that displays battery life, treatment time, a countdown timer, etc.) or a physical mechanism that automatically facilitates decoupling of the needleless connector 370 from the apparatus 300 after the sterilizing procedure. Display may include an indication that the sterilization treatment has been performed. This indication may be an image, such as a barcode or shape that can be scanned by the practitioner to register treatment to an electronic medical record. This indicator may only appear for a limited amount of time such as between 5 seconds and 1 minute and may include information such as time performed, duration of treatment, etc. In another embodiment, the apparatus may transmit such information (e.g., via a hard-line connection or wireless connection) to servers that store electronic medical records.

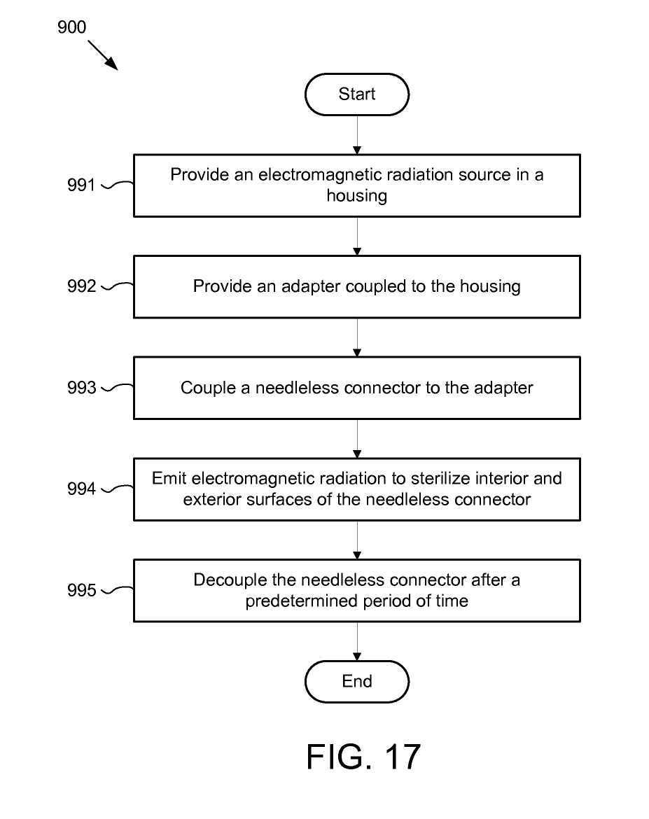

FIG. 17 is a schematic flowchart diagram of a method 900 for using the apparatus, according to one embodiment. The method 900 includes providing an electromagnetic radiation source positioned within a housing at 991. The method 900 further includes providing an adapter that is coupled to the housing and that spans an opening in the housing at 992. The adapter may have a cavity portion that is shaped to receive at least a portion of the needleless connector and the adapter may be made from a translucent material. The method 900 further includes coupling the needleless connector to the cavity portion of the adapter at 993 and subsequently emitting electromagnetic radiation from the electromagnetic radiation source for a predetermined period of time to sterilize both interior and exterior surfaces of the needleless connector at 994. During emission, electromagnetic radiation propagates through and is refracted by the translucent material of the adapter. After emitting the electromagnetic radiation for the period of time, the method 900 includes decoupling the needleless connector from the cavity portion of the adapter at 995. The method 900 may further include displaying a treatment verification symbol.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

The subject matter of the present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. An adapter for sterilizing a needleless connector comprising interior and exterior surfaces, the adapter comprising:
a proximal portion that is coupleable in electromagnetic radiation receiving communication with an electromagnetic radiation source; and
a cavity portion that is coupleable with the needleless connector and shaped to receive at least a portion of the needleless connector;
wherein the proximal portion and the cavity portion are made from a translucent material, wherein electromagnetic radiation from the electromagnetic radiation source propagates through and is refracted by the translucent material to sterilize both interior and exterior surfaces of the needleless connector.

2. The adapter of claim 1, wherein the translucent material is at least one of silica-derived material and fluoropolymers.

3. The adapter of claim 1, wherein the cavity portion comprises a male luer-lock fitting.

4. The adapter of claim 1, wherein the cavity portion comprises an annular ridge, an annular trench, and a protrusion, wherein the annular trench is between the annular ridge and the protrusion.

5. The adapter of claim 4, wherein as the needleless connector is coupled to the cavity portion of the adapter, the protrusion actuates a valve mechanism of the needleless connector to expose an interior surface of the needleless connector to the electromagnetic radiation.

6. The adapter of claim 5, wherein as the valve mechanism is actuated, the interior surfaces of the needleless connector are isolated from atmosphere external to the adapter.

7. The adapter of claim 4, wherein a distal surface of the annular ridge has a first surface roughness, and respective surfaces of the annular trench and the protrusion have a second surface roughness, the first surface roughness being lower than the second surface roughness.

8. An apparatus for sterilizing a needleless connector comprising interior and exterior surfaces, the apparatus comprising:
a housing comprising an opening;
an electromagnetic radiation source in the housing, wherein the electromagnetic radiation source is operable to emit electromagnetic radiation toward the opening; and
an adapter coupled to the housing and spanning the opening, wherein the adapter comprises a cavity portion that is coupleable with the needleless connector and that is shaped to receive at least a portion of the needleless connector, wherein the adapter is made from a translucent material and the electromagnetic radiation propagates through and is refracted by the translucent material to sterilize both interior and exterior surfaces of the needleless connector.

9. The apparatus of claim 8, wherein the cavity portion comprises an annular ridge, an annular trench, and a protrusion, wherein the annular trench is between the annular ridge and the protrusion.

10. The apparatus of claim 9, further comprising a disposable tip detachably coupleable to a distal end of the adapter, wherein the disposable tip is shaped to complement a shape of the distal end of the adaptor.

11. The apparatus of claim 10, wherein the disposable tip comprises a first region and a second region, wherein the first region substantially blocks the electromagnetic radiation and the second region substantially transmits the electromagnetic radiation, wherein the first region covers a distal surface of the annular ridge and the second region covers the annular trench and the protrusion.

12. The apparatus of claim 10, wherein the translucent material is a first translucent material, the disposable tip being made from a second translucent material that is different than the first translucent material.

13. The apparatus of claim 9, wherein as the needleless connector is coupled to the cavity portion of the adapter, the protrusion actuates a valve mechanism of the needleless connector to expose an interior surface of the needleless connector to the electromagnetic radiation, wherein as the valve mechanism is actuated, the interior surfaces of the needleless connector are isolated from atmosphere external to the adapter.

14. The apparatus of claim 8, wherein the translucent material is selected from the group consisting of: quartz, silica-derivatives, and fluoropolymers.

15. The apparatus of claim 8, wherein the cavity portion comprises a male luer-lock fitting.

16. The apparatus of claim 8, wherein the electromagnetic radiation source is an ultraviolet light source and the electromagnetic radiation is ultraviolet light that is non-visible to a human-eye, the apparatus further comprising a visible light source coupled to the housing, wherein visible light is emitted from the visible light source when ultraviolet light is emitted from the ultraviolet light source.

17. The apparatus of claim 8, further comprising:
a sensor that verifies a proper connection between a needleless connector and the cavity portion of the adapter; and
a controller that prevents emission of electromagnetic radiation from the electromagnetic radiation source unless the connection sensor verifies a proper connection between a needleless connector and the cavity portion of the adapter.

18. A method for sterilizing a needleless connector, comprising:
providing an electromagnetic radiation source positioned within a housing, wherein the housing comprises an opening;
providing an adapter coupled to the housing and spanning the opening, wherein the adapter comprises a cavity portion that is shaped to receive at least a portion of the needleless connector, wherein the adapter is made from a translucent material;
coupling the needleless connector to the cavity portion of the adapter;
after coupling the needleless connector to the cavity portion of the adapter, emitting electromagnetic radiation from the electromagnetic radiation source for a predetermined period of time to sterilize both interior and exterior surfaces of the needleless connector, wherein the electromagnetic radiation propagates through and is refracted by the translucent material of the adapter; and
after emitting the electromagnetic radiation for the predetermined period of time; decoupling the needleless connector from the cavity portion of the adapter.

19. The method of claim 18, further comprising verifying, by a connection sensor and a controller, a proper connection between the needleless connector and the cavity portion of the adapter before emitting the electromagnetic radiation.

20. The method of claim 18, wherein the predetermined period of time is set by a controller based on known sterilization requirements.
